(19) United States
(12) Patent Application Publication
(10) Pub. No.: US 2008/0009063 A1
(43) Pub. Date: Jan. 10, 2008

(54) SUBSTRATE FOR CELL CULTURE

(75) Inventors: Teruo Okano, Chiba (JP); Masayuki Yamato, Tokyo (JP); Yoshikatsu Akiyama, Tokyo (JP); Masanao Watanabe, Tokyo (JP); Hiroyuki Naganuma, Tokyo (JP); Masami Nara, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: Teruo OKANO, Ichikawa-shi (JP); DAI NIPPON PRINTING CO., LTD., Shinjuku-ku (JP)

(21) Appl. No.: 11/773,127

(22) Filed: Jul. 3, 2007

(30) Foreign Application Priority Data
Jul. 5, 2006 (JP) 2006-185666

(51) Int. Cl. C12N 5/02

(52) U.S. Cl. 435/402

(57) ABSTRACT

An object of the present invention is to provide a cell culture substrate capable of quickly forming a cell sheet and easily removing the cell sheet after formed. The present invention relates to a cell culture substrate for forming a cell sheet by culturing cells, comprising a plurality of projections each having a top face and depressions formed between the projections, in which the depressions each have an opening whose dimensions are too small for a cell to be cultured to enter and the cell sheet is removable. The present invention further relates to a method of preparing a cell sheet using the substrate and a cell sheet prepared by the method.

### BACKGROUND OF THE INVENTION

1. Field of the Invention

The present invention relates to a cell culture substrate for forming a cell sheet.

2. Background Art

The cell sheet is a sheet-form cell aggregate, in which cells are connected via intercellular junction to form at least a single layer, used in regenerative medicine. The cell sheet can be obtained by culturing cells on a substrate such as a Petri dish. However, the cell sheet formed on the substrate is strongly bonded to the surface of the substrate via adhesive molecules. Therefore, it is not easy to remove the cell sheet from the substrate quickly without breaking the cell-to-cell bonding.

In the circumstances, studies have been so far made on methods of efficiently removing a cell sheet from a cell culture substrate. The removing methods can be classified into two groups. In the first method, the bonding between a substrate and cells is weakened by an enzymatic reaction. In the second method, a substrate whose cell adhesive strength is weak or varies is used.

To explain more specifically, in the first method, enzymes such as protease (proteolysis enzyme) and collagenase (collagenolytic enzyme) are used to decompose proteins constituting intercellular adhesion molecules (involved in tight junction, adhesive junction, desmosome junction, a gap junction and hemi desmosome junction), collagen connective tissue surrounding a cultured product, and extracellular matrix (ECM) formed between cells and a substrate. In this method, not only cell-substrate bonding but also cell-cell bonding is weakened. The first method has long been used in the field of cell culture. Since the binding substances that are decomposed by this method are those produced in cells, tissues and organs to be cultured, they can be regenerated in predetermined conditions and period after a cell sheet is removed.

Nevertheless, the first method has problems: long time is required for regenerating the binding substances and the cell sheet formed by this method is more or less damaged. Thus, this method is not desirable for producing a cell sheet for use in regenerative medicine.

Under these circumstances, the second method has been newly developed.

The substrates having a weak cell adhesive strength are, for example, disclosed in JP Patent Publication (Kokai) Nos. 2004-170935A and 2005-168494A. These documents disclose a substrate having fine columnar projections called nanopillars on the surface and a technique for culturing cells on the substrate. In this method, since the substrate and a material to be cultured are in contact with each other only at an extremely small area, it is easy to remove and recover a cultured product with little damage.

Nevertheless, as is described in M. J. Dalby et al., Biomaterials 25 (2004) 5415-5422; and C. C. Berry et al., Biomaterials 25 (2004) 5781-5788, cell adhesion and the behavior of adhesion cells differ between flat-surface adhesion and irregular-surface adhesion. Culturing on the nanopillars has problems: the adhesion of cells and extension of a cell sheet on the nanopillars are slow and pseudopodium extends from a cell surface. In addition, when depressions of the irregular substrate have a width of 20 μm or more, cells enter the depressions.

The present inventors have developed a substrate whose cell adhesive strength varies, that is, a substrate having a coating of a temperature responsive polymer on the cell proliferation surface thereof (JP patent Publication (Kokoku) No. 6-104061B (1994)). The temperature responsive polymer is most preferably used for changing cell adhesion strength. Besides this, a pH responsive polymer and an ion responsive polymer can be used. JP Patent Publication (Kokai) Nos. 2004-170935A and 2005-168494A describes that a temperature responsive polymer is used in combination with culture using nanopillers. The use of a temperature responsive polymer in cell culture is also mentioned in Patent Publication (Kokai) No. 2005-27532A.

However, even if a conventional responsive polymer is used, the removal rate of a cell sheet is still insufficient. In a conventional technique, it takes 1 to 3 hours to remove a cell sheet. If the cell sheet can be removed much faster, for example, within 30 minutes, the cell sheet can be used at a medical operation site since the removal of the cell sheet can be finished within 30 minutes from starting. Consequently, more appropriate treatment can be ensured.

The present inventors obtained a cell sheet by use of a substrate formed of a porous membrane film having a temperature responsive polymer applied thereon, as described in O. H. Kwon, J. Biomed. Mater. Res., (2000) Apr.; 50(l):82-9. They confirmed that the cell sheet can be removed and recovered faster than that formed on a nonporous smooth membrane film substrate.

However, the removal and recover of a cell sheet by the method of O. H. Kwon is not yet sufficient in view of speed.

Note that the present applicant possesses many techniques in the fields of a photoresist material and microfabrication. JP patent Publication (Kokai) No. 2005-84561A describes reproductions such as a sub-micron diffraction grating and a method of manufacturing the same.

There are documents relevant to the present invention other than set forth above. The documents are:

JP Patent No. 312660,

JP Patent No. 3491917,

JP Patent Publication (Kokai) No. 9-12651A (1997),

JP Patent Publication (Kokai) No. 10-248557A (1998),

JP Patent Publication (Kokai) No. 11-349643A (19991),

JP Patent Publication (Kokai) 2001-329183A,

JP Patent Publication (Kokai) 2002-18270A,

JP Patent Publication (Kokai) No. 5-244938A (1993), and

International Publication WO 01/068799.

### SUMMARY OF THE INVENTION

As described above, conventional cell culture substrates for preparing a cell sheet have following problems: a cell sheet may be damaged when it is removed from a substrate; a cell sheet cannot be formed uniformly on a substrate; the formation rate of a cell sheet on a substrate is low; and long time is required to remove a cell sheet from a substrate.

An object of the present invention is to provide a cell culture substrate successfully overcoming the aforementioned problems.

The present application includes the following inventions.

(1) A cell culture substrate for forming a cell sheet by culturing cells comprising a plurality of projections each having a top face and depressions formed between the projections, in which the depressions each have an opening whose dimensions are too small for the cell to be cultured to enter. The cell culture substrate is characterized in that the cell sheet is preferably removable.

(2) The cell culture substrate according to item (1), in which the top face is coated with at least one type of polymer selected from the group consisting of a temperature responsive polymer, pH responsive polymer and ion responsive polymer.

(3) The cell culture substrate according to item (1) or (2), in which the dimensions of the opening of each of the depressions are smaller than the outer dimensions of the cell to be cultured.

(4) The cell culture substrate according to any one of items (1) to (3), in which the total area of the openings of the depressions is 10 to 60% of the sum of the total area of the top faces and the total area of the openings of the depressions.

(5) The cell culture substrate according to any one of items (1) to (4), in which the opening of each of the depressions has a width of 0.1 to 10 μm.

(6) The cell culture substrate according to any one of items (1) to (5), in which the opening of each of the depressions has a depth of not less than 0.01 [μm.

(7) The cell culture substrate according to any one of items (1) to (6), in which the dimensions of the top face are equal to or larger than the outer dimensions of the cell to be cultured.

(8) The cell culture substrate according to any one of items (1) to (7), in which the width of the top face is 1 to 20 μm.

(9) The cell culture substrate according to any one of items (1) to (8), in which the depressions form a grid-form groove.

(10) The cell culture substrate according to any one of items (1) to (8), wherein the depressions form a plurality of linear grooves.

(11) A method of forming a cell sheet comprising by culturing cells by use of the cell culture substrate according to any one of items (1) to (10).

(12) A cell sheet prepared by the method of item (11).

When a cell sheet is prepared by using a cell culture substrate according to the present invention, the cell sheet can be removed quickly. Since the time required for removing the cell sheet is short, the cell sheet is virtually not denatured during a removal operation.

The cell sheet prepared by using a cell culture substrate according to the present invention, since an adhesion factor present on the surface of the cell sheet is not damaged, can be suitably used in regenerative medicine.

This specification encompasses the contents described in the claims specification and/or drawings of Japanese Patent Application No. 2006-185666 which the priority of this application is based on.

### BRIEF DESCRIPTION OF THE DRAWINGS

FIG. 1a is a top view of a cell culture substrate (100) having projections (101) and depressions (103) formed between the projections and constitute a grid groove as a whole;

FIG. 1b is a perspective view of the projections (101) and depressions (103) of the cell culture substrate (100) partly magnified;

FIG. 1c is an illustration indicating definitions of a groove width, pitch and depth;

FIG. 2 is a view showing another preferable embodiment of the cell culture substrate of the present invention;

FIG. 3 is a view showing another preferable embodiment of the cell culture substrate of the present invention;

FIG. 4 is a top view of a cell culture substrate according to a Reference Example 1;

FIG. 5 is a top view of a cell culture substrate according to a Reference Example 2;

FIG. 6a is a top view of a cell culture substrate according to Comparative Example;

FIG. 6b is a sectional view of the cell culture substrate according to Comparative Example;

FIG. 7 is a graph showing a removal rate of a confluent-cell sheet versus time;

FIG. 8a shows photographs showing formation of the extracellular matrix of cells cultured by use of the substrate according to Example 1 and removal of the cell sheet;

FIG. 8b shows photographs showing formation of the extracellular matrix of cells cultured by use of the substrate according to Example 5 and removal of the cell sheet; and

FIG. 8c shows photographs showing formation of the extracellular matrix of cells cultured by use of the substrate according to Comparative Example and removal of the cell sheet.

### DETAILED DESCRIPTION OF THE PREFERRED EMBODIMENTS

Typical embodiments of a cell culture substrate according to the present invention, which has a plurality of projections each having a top face and depressions formed between the projections, will be explained in detail with reference to the drawings.

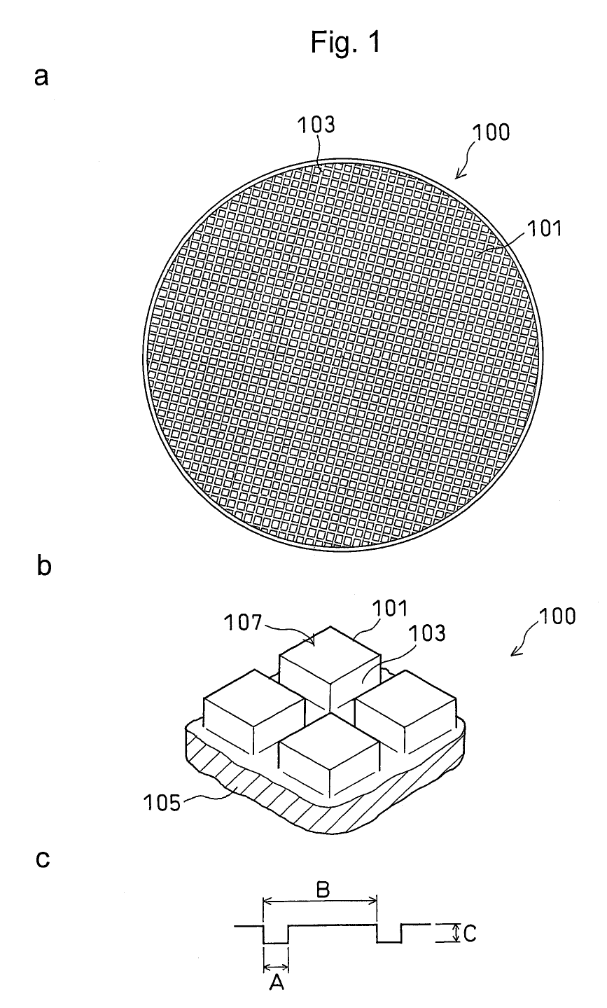

FIG. 1a shows a top view of a cell culture substrate (100) having projections (101) and depressions (103) that are formed between the projections and constitute a grid groove; FIG. 1b shows a perspective view of the projections and depressions of the cell culture substrate (100) partly magnified. The projections (101) are formed so as to protrude from a substrate base (105). The depressions (103) form a groove using the substrate base (105) as the bottom thereof. The projections (101) each have a smooth square top face (107). Cells proliferate so as to cross over the depressions and cover a plurality of top faces to form a cell sheet.

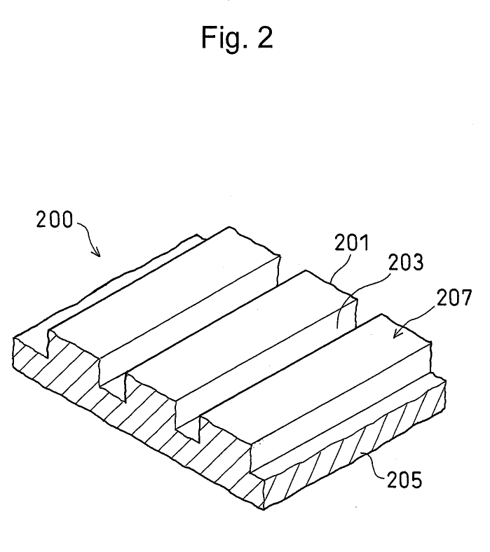

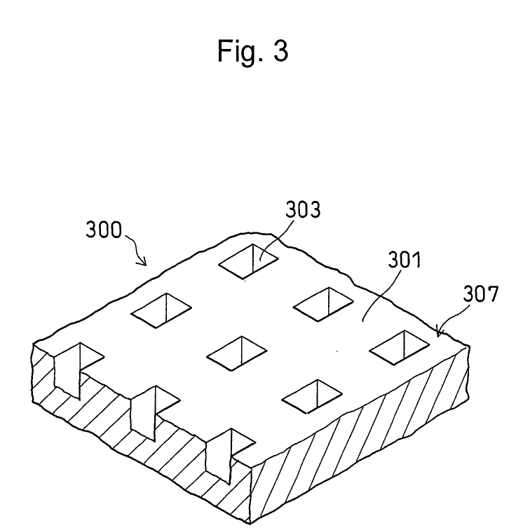

FIGS. 2 and 3 show other embodiments of the cell culture substrate of the present invention.

A cell culture substrate (200l ) is constituted of a plurality of linear projections (201), which protrude from a substrate base (205) and are arranged in parallel, and a plurality of depressions (203), which are formed between the projecting lines and form a linear groove. Each of the linear projections (201) has a smooth band-form top face (207). Cells proliferate so as to cross over the depressions and cover a plurality of top faces to form a cell sheet, in the same manner as in the embodiment shown in FIG. 1.

A cell culture substrate according to the present invention essentially requires “a plurality of projections” as a constituent feature. The projections may be partly connected with each other as long as depressions can be formed between them. For example, it is possible that the projections (101) shown in FIG. 1b are partly connected with the adjacent projections to form a cell culture substrate (300) as shown in FIG. 3. The cell culture substrate (300) is also a preferred embodiment of the present invention. In this embodiment, the depressions (303) each has a square opening with a bottom. In the cell culture substrate (300), cells proliferate so as to cross over the depressions (303) and cover a top faces (307) to form a cell sheet.

In the present invention, the top faces (107, 207, 307) of the projections of a same substrate are preferably positioned in the same plane.

Examples of a material for a cell culture substrate according to the present invention include, but not limited to, glasses, plastics, ceramics and metals, which are usually used for cell culture. Any material may be used as long as cells can be cultured on it. Furthermore, an optional layer may be provided on the surface or in the middle of the substrate, or an optional treatment may be applied to the substrate as long as it does not interfere with an object of the present invention. For example, a hydrophilic treatment such as ozone treatment, plasma treatment or sputtering may be applied to the surface of the substrate.

A cell culture substrate according to the present invention may be manufactured by various types of microfabrication techniques known to a person skilled in the art. Examples of the microfabrication techniques include, but not limited to, lithographic techniques as shown in JP Patent Publication (Kokai) No. 2005-84561A.

A cell culture substrate according to the present invention may take any shape as a whole as long as it has a plurality of projections each having a top face serving as a scaffold for the cells to be cultured and depressions formed between the projections. For example, a cell culture substrate according to the present invention can be obtained by forming any one of the micropatterns as shown in FIGS. 1 to 3 at the bottom of a Petri dish or a culture vessel commonly used in the art.

The whole size of a cell culture substrate according to the present invention can be appropriately selected depending upon the size of the cell sheet to be prepared.

The top face of each of the projections is preferably coated with at least one type of polymer selected from the group consisting of a temperature responsive polymer, pH responsive polymer and ion responsive polymer in order to facilitate removal of a cell sheet, and more preferably, coated with a temperature responsive polymer. Note that the coating with such a responsive polymer may be applied to the faces other than the top faces of the projections, for example, side faces of the projections and bottom faces of the depressions.

The temperature responsive polymer that can be suitably used in the present invention is preferred to exhibit hydrophobicity at a cell-culturing temperature (generally, about 37° C.) and exhibit hydrophilicity at a temperature for recovering a cell sheet after culture. Note that the temperature (critical solution temperature (T) to water), at which the nature of the temperature responsive polymer changes from hydrophobicity to hydrophilicity, is not particularly limited; however, in view of convenience of recovering a cell sheet after culture, the temperature T is preferably lower than the temperature for culturing cells. Since such a temperature responsive polymer component provides (ensures) the scaffold of cells (cell adhesive surface) during cell culturing, cell culture can be efficiently performed. On the other hand, when a cell sheet is removed and recovered after culture, a hydrophobic part of the cell culture substrate is changed to be hydrophilic, thereby separating the cell sheet obtained after culture from the cell culture substrate. In this manner, recovery of the cell sheet can be further facilitated.

Specific examples of the temperature responsive polymer that may be suitably used in the present invention include polymers having a critical solution temperature (T) of 0° C. to 80° C., and more preferably, 0° C. to 50° C. This is because when T exceeds 80° C., cells may die. In contrast, when T is lower than 0° C., a cell proliferation rate extremely decreases or cells die, in general. Examples of the suitable polymer include polymers described in JP Patent Publication (Kokoku) No 6-104061A (1994), such as poly-N-isopropylacrylamide (T=32° C.), poly-N-n-propylacrylamide (T=21° C.), poly-N-n-propylmethacrylamide (T=32° C.), poly-N-ethoxyethylacrylamide (T=about 35° C.), poly-N-tetrahydrofurfurylacrylamide (T=about 28° C.), poly-N-tetrahydrofurfurylmethacrylamide (T=about 35° C.) and poly-N,N-diethylacrylamide (T=32° C.). Examples of other polymers include alkyl substituted cellulose derivatives such as poly-N-ethylacrylamide, poly-N-isopropylmethacrylamide, poly-N-cyclopropylacrylamide, poly-N-cyclopropylmethacrylamide, poly-N-acryloylpyrrolidine, poly-N-acryloylpiperidine, polymethylvinylether, methylcellulose, ethylcellulose, and hydroxypropylcellulose; and polyalkyleneoxide block copolymers represented by a block copolymer of a polypropylene oxide and a polyethylene oxide. These polymers can be prepared by homopolymerization or copolymerization of monomers which if homopolymerized would result in homopolymers having T=0 to 80° C. Examples of the monomers include (metha) acrylamide compounds, N-(or N,N-di) alkyl substituted (metha) acrylamide compounds, (metha) acrylamide derivatives having a cyclic group and vinyl ether derivatives. These may be used singly or in combination. In the cases where T needs to be controlled depending upon the type of cells to be proliferated, where the interaction between a coating substance and a cell culture substrate needs to be improved, and where the balance of hydrophilicity and hydrophobicity of the cell culture substrate needs to be controlled, other monomers besides the aforementioned monomers may be added and copolymerized. Furthermore, use may be made of a graft or block copolymer between any one of the aforementioned polymers and another polymer, a mixture of a polymer according to the present invention and other polymer(s). Polymers may be crosslinked as long as they do not lose their inherent natures.

A pH responsive polymer and ion responsive polymer may be appropriately selected in accordance with the cell sheet to be prepared.

The coating amount of responsive polymer to be applied to the top face of a projection is 5 to 80 μg/cm², and preferably, 6 to 40μg/cm². When the coating amount of polymer exceeds 80 μg/cm², cells cannot adhere onto the surface of a cell culture substrate. In contrast, when the coating amount of polymer is less than 5 μg/cm², cells are cultured to form a single layer and form no tissue. In addition, it becomes difficult to remove and recover the cultured cells from the substrate. The coating amount of polymer can be obtained by Attenuated Total Reflectance Fourier Transform Infrared Spectroscopy (ATR-FT-IR), staining of a coating portion or non-coating portion with a dye, analysis of fluorescent staining, and surface analysis based on contact angle measurement, singly or in combination.

A polymer may be applied onto the surface of the top face of the projections by chemical and physical methods (described later) singly or in combination. When a monomer as mentioned above is used for coating, the monomer may take any state such as a gaseous, liquid and solid state. When a polymer is used, the polymer may take either liquid or solid state. These monomers or polymers may be chemically bonded to the surface of the top face of the projections by electron beam irradiation (EB), γ-ray irradiation, UV irradiation, plasma treatment and corona treatment. Furthermore, when the material for the surface of the top face and a coating material have appropriate functional groups having reactivity, they are bonded by a general organic reaction such as a radical reaction and ionic reaction. When they are bonded by physical interaction, a coating material may be physically adsorbed onto the surface of a top face by means of coating or kneading, solely by itself or with a matrix (having good compatibility with the material for the top face) used as a medium. However, the bonding method is not limited to these.

The responsive-polymer coating layer may be provided over the entire surface of projections and depressions after a micro-pattern is formed or provided only over the top surface of the projections. Alternatively, after a polymer layer and a micro-pattern are separately prepared, they may be bonded to each other. The responsive-polymer coating layer may be provided via an intermediately layer as long as the layer does not affect a micro-pattern. The formation of a micro-pattern and polymer coating may be performed at the same time.

One of the characteristics of a cell culture substrate according to the present invention resides in that the dimensions of the opening portion of depressions are too small for the cell to be cultured to enter. Since a cell cannot enter the depressions, the formation of a cell sheet cannot be interrupted by the presence of depressions, with the result that the cell sheet can be formed smoothly so as to cover the top faces of projections. In addition, the removal rate of the cell sheet can be facilitated. In particular, the dimensions of the opening of depressions are preferably smaller than the outer dimensions of the cultured cells. The phrase “smaller than the outer dimensions of the cultured cells” means that the length (width) of the shorter side of the opening of a depression is shorter than the length (width) of the shorter side of the outer dimensions of the cell cultured. The “length of the shorter side of the opening of a depression” refers to the minimum length of the shorter sides of the openings when they differ in length. For example, the width of the opening is preferably 0.1 to 10 μm, and more preferably, 0.3 to 2.5 μm; however, it may be appropriately selected depending upon the type of cell to be cultured. The depressions store water and may play a role of supplying water to a cell-sheet removal site when the cell sheet is removed. Therefore, the depressions preferably have a sufficient depth to store water. More specifically, the depressions each preferably have a depth of 0.01 μm or more. The upper limit of the depth is not particularly determined; however, in consideration of an efficiency of processing, about 20 μm is preferable as the upper limit.

As is explained in the section of the Background of Art, when a cell sheet is formed of a microporous film having a temperature responsive polymer which is graft-bonded onto the surface thereof as is described in O. H. Kwon, J. Biomed. Mater. Res., (2000) Apr.; 50(1):82-9, it is impossible to remove the cell sheet quickly. This phenomenon can be explained based on the results of Comparative Example described in the specification of the present application. To remove the cell sheet, water must be supplied to the interface between the cell culture substrate and a cell sheet; however, water cannot penetrate into a microporous film coated with a temperature responsive polymer in low-temperature conditions for removing the cell sheet. Therefore, when the cell sheet is formed on the microporous film, water is not sufficiently supplied onto the adhesion surface of the cell sheet. In this case, it may be difficult to remove the cell sheet. On the other hand, according to a cell culture substrate of the present invention, water present in depressions is supplied to the adhesion surface of a cell sheet. Therefore, the removal of the cell sheet can be carried out at high speed.

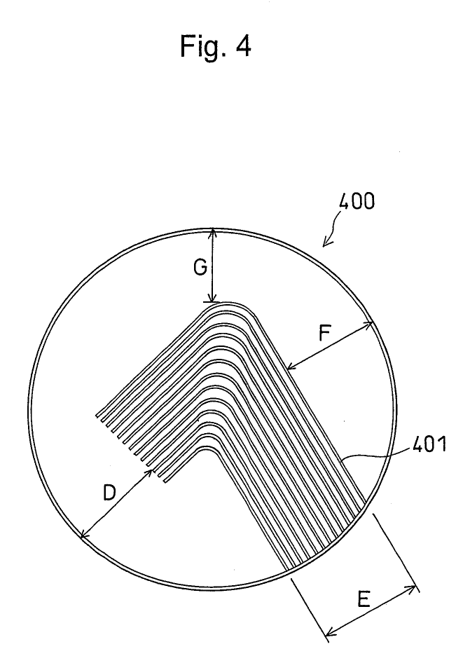

The shape of depressions is not particularly limited. It is typically preferable that they form a grid-form groove as shown in FIG. 1, a plurality of linear grooves arranged in parallel as shown in FIG. 2, or dents/holes having a square or rectangular opening as shown in FIG. 3. However, any types of depressions may be used as long as they can be formed between projections having a shape as described later. Note that a grid-form groove used in the present invention includes not only an orthogonal grid but also an oblique grid. Note that the linear groove includes not only a linear groove but also a curved groove as shown in FIG. 4. The sectional view of the short side of depressions is not limited to a shape of a rectangle lacking an upper side, as shown in FIGS. 1 to 3. Any shape may be used.

On a cell culture substrate according to the present invention, unlike the case of the cell culture performed on nanopillars described in JP Patent Publication (Kokai) Nos. 2004-170935A and 2005-168494A, a cell sheet is extended from a cell placed at initiation time of culture radially (in all directions) toward the edge of the cell culture substrate. As a result, a cell sheet having uniform quality can be obtained. This feature can be preferably attained by the following two characteristics of the invention: depressions each have an opening satisfying the aforementioned dimensions and projections each have a top face having a certain area satisfying the aforementioned dimensions, unlike nanopillars. The dimensions of the top face are preferably equal to or larger than the outer dimensions of the cell to be cultured. When the dimensions of a top face satisfy this range, the cultured cells exhibit the same behavior as that of the cells cultured in a smooth surface having no depressions. The phrase “the dimensions of the top face are preferably equal to or larger than the outer dimensions of a cell” means that the length (width) of the shorter side of the top face of the projections is equal to or longer than the length (width) of the shorter side of the outline of a cell. The “length (width) of the shorter side of the top face” refers to the minimum length thereof when the shorter sides of the top faces of projections vary in length. The width of the top face is, for example, 1 to 20 μm, and preferably, 2 to 15 μm; however, it may be appropriately selected depending upon the type of cell to be cultured. When the width of a top face is too broad, the area of the contact face between the top face and a cell sheet increases. As a result, the moving distance of water stored in depressions to a responsive polymer increases and long time is required for removing the cell sheet.

The shape of the top face of projections as viewed from the cell adhesion surface is not particularly limited; however, a circle, a polygon such as a triangle, square, pentagon or hexagon, or a narrow linear or curved band is typically preferable. In the sectional shape of the projections, the side line of the projections may be vertical, oblique, or curved outward or inward.

Another feature of a cell culture substrate according to the present invention resides in that a cell sheet can be quickly removed. The feature cannot be attained by use of a responsive polymer alone; however, it can be attained by projections whose top faces do not have an excessively large area and arrangement of depressions having minute openings at an appropriate ratio between the projections, in combination with the responsive polymer. When the ratio of the openings of depressions is high, it becomes easy to remove a cell sheet but the formation rate of the cell sheet becomes slow. In contrast, when the ratio of the openings is low, it becomes difficult to remove the cell sheet. In view of these problems, the ratio (%) of the total area of openings relative to the sum of the total area of top faces and the total area of openings is preferably 10 to 60%, more preferably 15 to 50%, and most preferably, 20 to 45%.

By using a cell culture substrate according to the present invention, a cell sheet can be prepared from various types of cells such as epithelial cells and endothelial cells constituting tissues and organs in a living body; skeletal muscle cells, smooth muscle cells, and cardiac muscle cells having contractility; neurons, glia cells and fibroblast cells constituting the nervous system; hepatic parenchymal cells, non-hepatic parenchymal cells and fat cells involved in the metabolism of a living body; cells having the differentiation potency such as stem cells present in various types of tissues; myeloblast cells and ES cells. The cell sheet thus prepared, since it keeps an intact adhesion factor on the surface, is suitable for use in regenerative medicine. The cell sheet can be further applied to a detection device such as a biosensor.

### EXAMPLES

### Preparation of Cell Culture Substrate

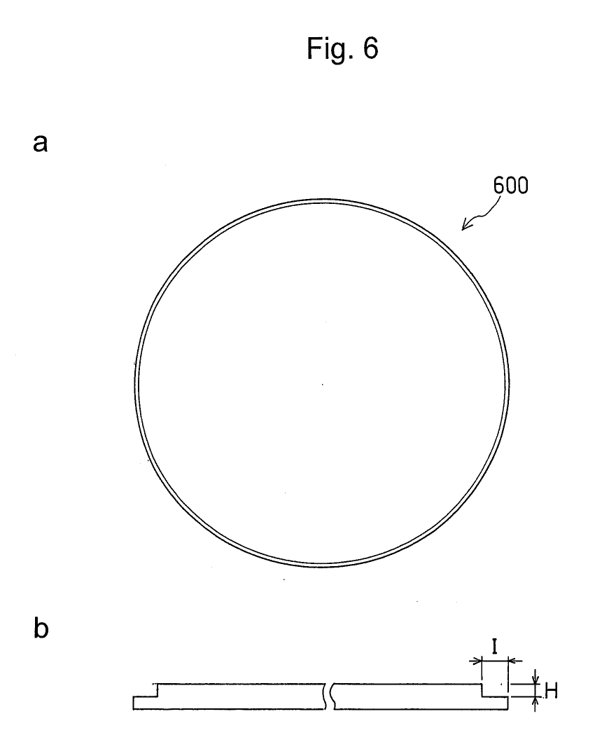

In Examples 1 to 6, cell culture substrates formed of projections having a square top face and a grid-form groove (as shown in FIG. 1) were prepared. More specifically, on a disk-form substrate of 13 mm in radius, micropatterns satisfying a groove width A (shown in FIG. 1c and Table 1) and pitches B (shown in FIG. 1c and Table 1) were formed. The depth of the grooves (see C in FIG. 1c) were set at a constant value of 0.5 μm. The cell culture substrates according to Examples 1 to 6 have the same stepped portion at the edge as shown in the sectional view of Comparative Example described later. The difference in height between the surface of the cell culture substrate and that of the stepped portion (see H in FIG. 6b) is 0.5 μm and the width of the stepped portion (see I in FIGS. 6a and 6b) is 200 μm.

As Reference Example 1, a cell culture substrate (400) as shown in FIG. 4 was formed. The cell culture substrate (400) partly has a plurality of L-shape grooves (401) arranged in parallel. More specifically, on a disk-form substrate of 13 mm in radius, a plurality of L-shape grooves (401) having a width of 1.5 μm and a depth of 0.5 μm were formed in parallel at pitches of 7.5 μm. The cell culture substrate according to Reference Example 1 has the same stepped portion at the edge as shown in the sectional view of Comparative Example described later. The difference in height between the surface of the cell culture substrate and that of the stepped portion (see H in FIG. 6b) is 0.5 μm and the width of the stepped portion (see I in FIGS. 6a and 6b) is 200 μm. In FIG. 4, the dimensions of the portions indicated by reference symbols D, E, F and G are 7.00 mm, 7.28 mm, 7.04 mm and 5.20 mm, respectively.

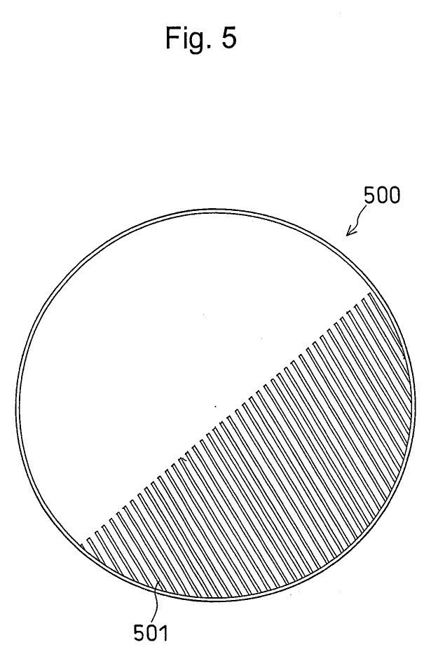

As Reference Example 2, a cell culture substrate (500) as shown in FIG. 5 was formed. The cell culture substrate (500) has a plurality of linear grooves (501) arranged in parallel in a half area of the substrate. More specifically, on a disk-form substrate of 13 mm in radius, a plurality of linear grooves (501) having a width of 1.5 μm and a depth of 0.5 μm were formed in parallel at pitches of 7.5 μm. The cell culture substrate according to Reference Example 2 has the same stepped portion at the edge as shown in the sectional view of Comparative Example described later. The difference in height between the surface of the cell culture substrate and that of the stepped portion (see H in FIG. 6b) is 0.5 μm and the width of the stepped portion (see I in FIGS. 6a and 6b) is 200 μm.

As Comparative Example, a cell culture substrate (600) having no micropattern was formed as shown in FIGS. 6a and 6b. More specifically, on a disk-form substrate of 13 mm in radius, a table-form portion having a smooth top was formed. The cell culture substrate according to Comparative Example has a stepped portion at the edge as shown in a sectional view. The difference in height between the surface of the cell culture substrate and that of the stepped portion (see H in FIG. 6b) is 0.5 μm and the width of the stepped portion (see I in FIGS. 6a and 6b) is 200 μm.

TABLE 1

| | | | | (Areas of |
|---|---|---|---|---|
| | | | | groove- |
| | | | | openings/ |
| | | | | areas |
| | | | | of top |
| | | | | faces + |
| | | | | areas of |
| | | Groove | | groove- |
| | | width (A) | Pitch (B) | openings) × |
| | Pattern | (μm) | (μm) | 100 (%) |
| Example 1 | Grid | 3.0 | 15.0 | 36.0 |
| Example 2 | Grid | 2.0 | 8.0 | 43.8 |
| Example 3 | Grid | 1.5 | 7.5 | 36.0 |
| Example 4 | Grid | 1.5 | 6.0 | 43.8 |
| Example 5 | Grid | 1.5 | 13.5 | 21.0 |
| Example 6 | Grid | 1.0 | 5.5 | 33.1 |
| Reference | L-shape grooves in | 1.5 | 7.5 | |
| Example 1 | part | | | |
| Reference | Linear grooves | 1.5 | 7.5 | |
| Example 2 | arranged in half area | | | |
| Comparative | None | — | — | |
| Example | | | | |

In the cell culture substrates of Examples 1 to 6, a micropattern was formed in accordance with the procedure described in Example 2 of JP Patent Publication (Kokai) No. 2005-84561A. More specifically, a soda glass disk of 13 mm in radius was used as a base substrate (105). On the base substrate (105), projections (101) were formed of a molding resin composition as described below. The procedure will be more specifically described.

On a synthetic quartz board of 6.35 mm thick, a Cr layer of 0.11 μm thick was formed. On the Cr layer, a photoresist layer was formed. A resist pattern was formed by light exposure in patterned manner, developed and used for etching the Cr layer. After etching the Cr layer, the quartz substrate was dry-etched, and then, the resist pattern was dissolved and removed. In this manner, grooves (depressions) having a depth (0.5 μm) and a width as shown in Table 1 were formed at pitches as shown in Table 1 so as to correspond projections having a square top face arranged at regular intervals. Thereafter, the Cr layer was removed by acid etching. The resultant construct was further subjected to a treatment with hydrofluoric acid to obtain a molding pattern.

As the UV-sensitive molding resin composition, the following composition was prepared.

TABLE 2

| Molding resin | Content | |
|---|---|---|
| Urethane acrylate (GOHSELAC UV-7500B (trade name) | 35 | parts |
| manufactured by Nippon Synthetic Chemistry Industry Co., | | |
| Ltd.) | | |
| 1,6-hexanedioldiacrylate (manufactured by Nippon | 35 | parts |
| Shokubai Co., Ltd.) | | |
| Pentaerythritoltriacrylate (manufactured by Toagosei | 10 | parts |
| Co., Ltd.) | | |
| Vinylpyrrolidone (manufactured by Nippon Shokubai | 15 | parts |
| Co., Ltd.) | | |
| 1-hydroxycyclohexylphenylketone (Irugacure 184 (trade | 2 | parts |
| name) manufactured by Ciba Specialty Chemicals) | | |
| Benzophenone (manufactured by Nakarai Tesque Inc.) | 2 | parts |
| Alcohol-denatured silicone oil (TSF4570 (trade name) | 1 | part |
| manufactured by GE Toshiba Silicone Co., Ltd.) | | |

The molding resin having the composition shown in Table 2 was added dropwise onto the side of the molding pattern (produced as described above) having grooves (depressions). A soda glass substrate of 1.1 mm thick having an anchoring treatment previously applied thereto, was stacked on the molding resin such that the treatment surface of the soda glass substrate faced the molding resin. UV rays of 170 mJ/cm² (365 nm) were applied to the molding pattern by use of a high-pressure mercury-vapor lamp to harden the molding resin composition placed between the molding pattern and the soda glass substrate. Thereafter, the molding pattern body was removed to obtain a micropattern as shown in FIG. 1. The micropattern body thus obtained was perfect having no deletion in pattern. Furthermore, hardened molding resin did not remain in the grooves of the molding pattern after separation.

The substrates of Reference Examples 1 and 2 were produced in the same manner as in Examples 1 to 6 except that the shapes of projections differed from those of Examples.

The substrate of Comparative Example was produced in the same manner as in Examples 1 to 6 except that a micro pattern was not provided.

Coating with a Temperature Responsive Polymer

The substrate prepared above was coated with a temperature responsive polymer in the following procedure. The irregular surface of the substrate was previously treated with oxygen plasma to render the surface clean and wet.

A 40 wt % N-isopropylacrylamide solution in isopropyl alcohol was prepared. Onto the irregular surface of each of the substrates prepared above, 12 μl of the 40 wt % N-isopropylacrylamide solution in isopropyl alcohol was added and the substrate was irradiated with an electron beam of 30 Mrad. In this manner, the irregular surface of the substrate was coated with poly-N-isopropylacrylamide. After the electron-beam irradiation, the substrate was washed with ion exchange water to remove remaining monomer and free poly-N-isopropylacrylamide. The substrate was dried in a clean bench and sterilized with ethylene oxide (EO) gas, and then, deaeration was sufficiently performed to obtain a cell culture substrate.

Preparation of Cell Sheet on Cell Culture Substrate

Each of the cell culture substrates obtained above was placed on the bottom of a Petri-dish and the vascular endothelial cells taken from the bovine aorta were cultured on the cell culture substrate by a customary method using Dulbecco's modified Eagle medium (DMEM) containing 10% fetal calf serum (FCS) at 37° C. under 5% CO₂.

Five days after initiation of culture, a whole Petri dish containing the cell culture substrate having confluent vascular endothelial cells attached thereto was transferred to a chamber containing 5% CO₂ at 20° C. and placed under microscopic observation. At the same time, the entire Petri dish was taken by a digital video camera.

The adhesion state of cells and extension size of a cell-sheet did not greatly differ between cell culture substrates subjected to the experiment regardless of the presence or absence of irregularity and were satisfactory as a whole. No problem was found on time required for cells to reach a confluent state although the pattern (Example 1) having grooves of 3 μm width exhibits a half-day delay at maximum.

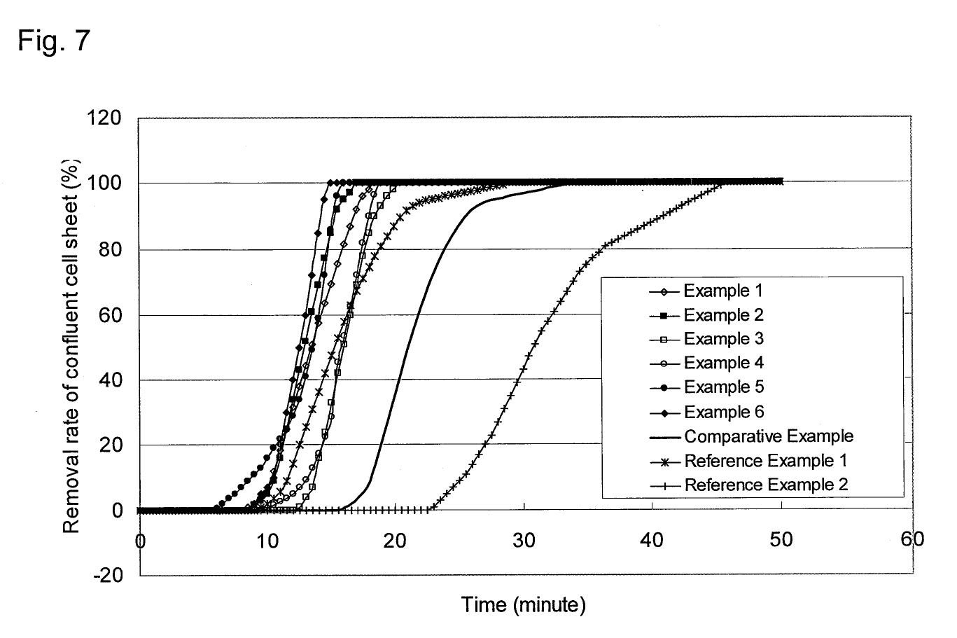

The degree of removal was evaluated based on the removal rate (%) of a confluent cell sheet versus time. The removal rate was obtained from image analysis of micrographs, time lapse photographs and pictures taken by a digital video camera. The results are shown in FIG. 7. As is apparent from the figure, a cell sheet was removed completely in Examples 1 to 6 faster than in Comparative Example. In Reference Examples 1 and 2, removal of a cell sheet started from a smooth portion and then removal in an irregular portion also started. The removal rate of the cell sheet from the irregular portion was relatively fast; however, it took long time to completely remove the cell sheet from the smooth portion. If a plurality of linear or curved grooves are provided in the entire surface of the substrate, the cell sheet may be completely removed at high speed similarly to the case of a grid-form groove.

In any one of the cell culture substrates used in the experiment, a cell sheet was completely removed with no cells remaining on the substrates. According to microscopic observation, a part of a cell was stuck at a groove only in the pattern of Example 1 having grooves of 3 μm width; however, the stuck portion of a cell was removed together with the cell sheet and incorporated into the cell sheet. However, even this phenomenon was not observed in other patterns.

Observation of Process for Forming Extracellular Matrix (ECM)

In the cell sheet prepared in Examples 1 and 5 and Comparative Example, the formation state of an extracellular matrix (hereinafter, referred to as an “ECM”) was observed. ECM was observed by staining a main component, fibronectin, with a fluorescent dye in accordance with the procedure described in “Materials and Methods” of A. Kushida et al., J. Biomed. Mater. Res., (1999) Jun.; 45(4):355-62. The subconfluent-state cell sheet obtained 3 days after initiation of culture, and the confluent-state cell sheet obtained 5 days after initiation of culture were subjected to fluorescent staining. The procedure of the fluorescent staining will be described below.

In the cell sheet preparation step mentioned above, the sheet-form cultured cells attached on a substrate was taken out 3 days and 5days after initiation of culture, and medium was removed from the sheets. Each of the cell sheets were washed twice with a phosphorus buffer solution (hereinafter, PBS) at 37° C., fixed with 4% paraformaldehyde/PBS for 20 minutes at 37° C., washed with PBS, 0.5%, TritonX-100/PBS and then, PBS. Thereafter, the cell sheet was fixed with 0.1% bovine serum albumin/PBS for one hour, allowed to bind to a primary antibody/bovine fibronectin rabbit polyclonal antibody, washed with PBS, allowed to bind to a secondary fluorescent antobody/Alexa488 conjugated anti-rabbit goat IgG, and washed with PBS.

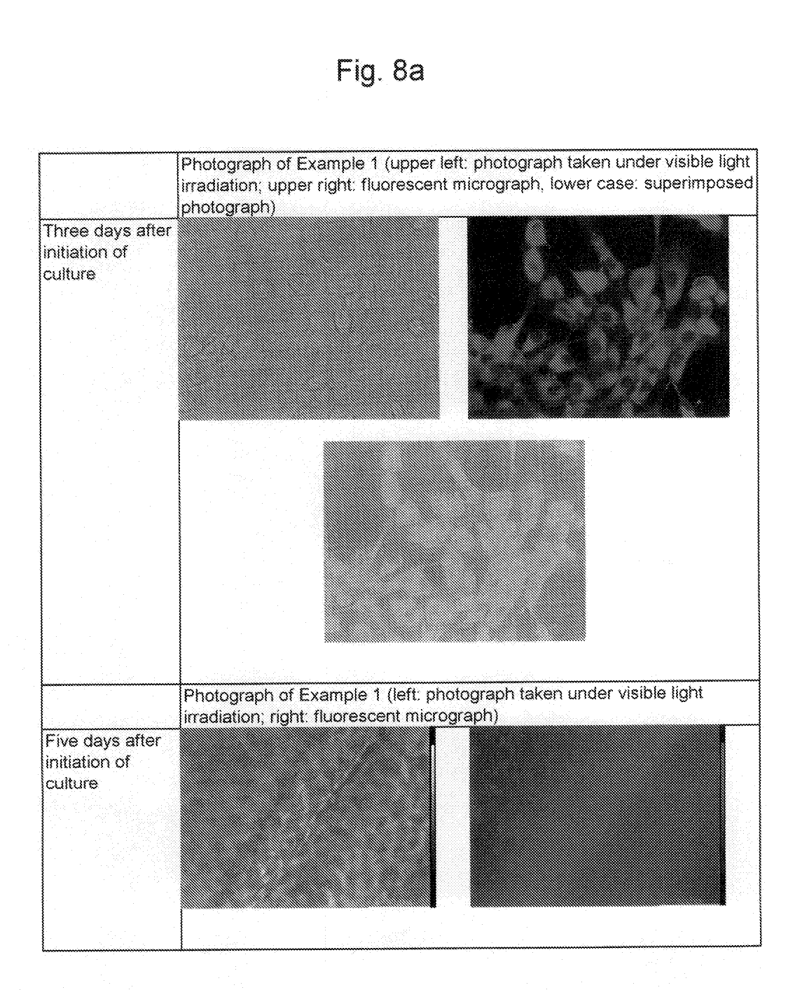

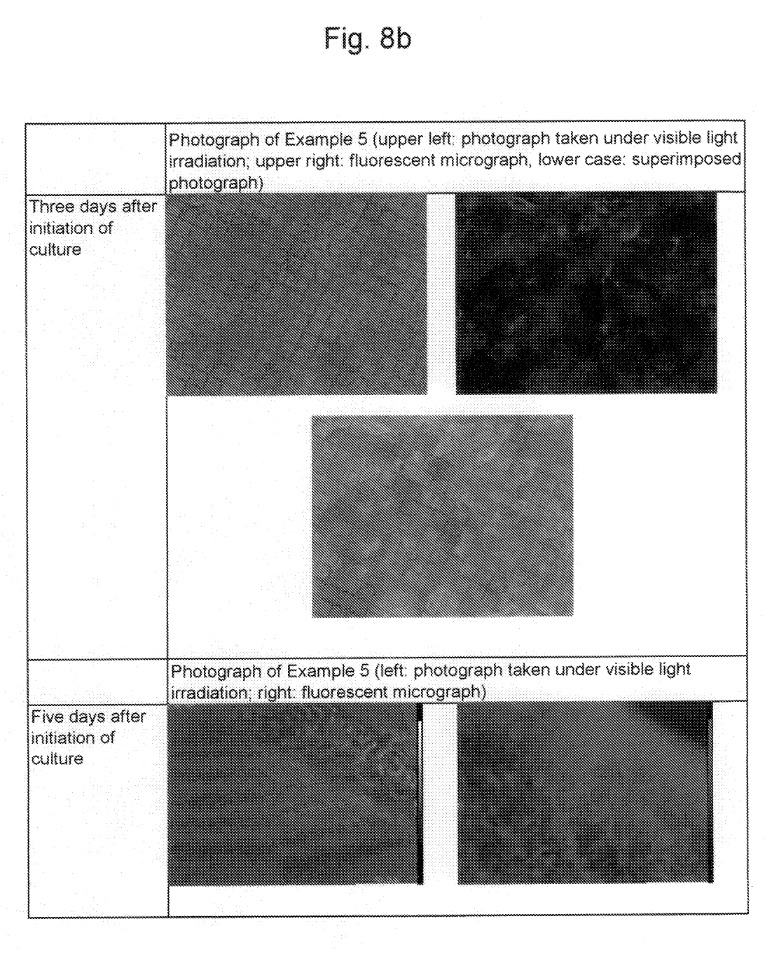

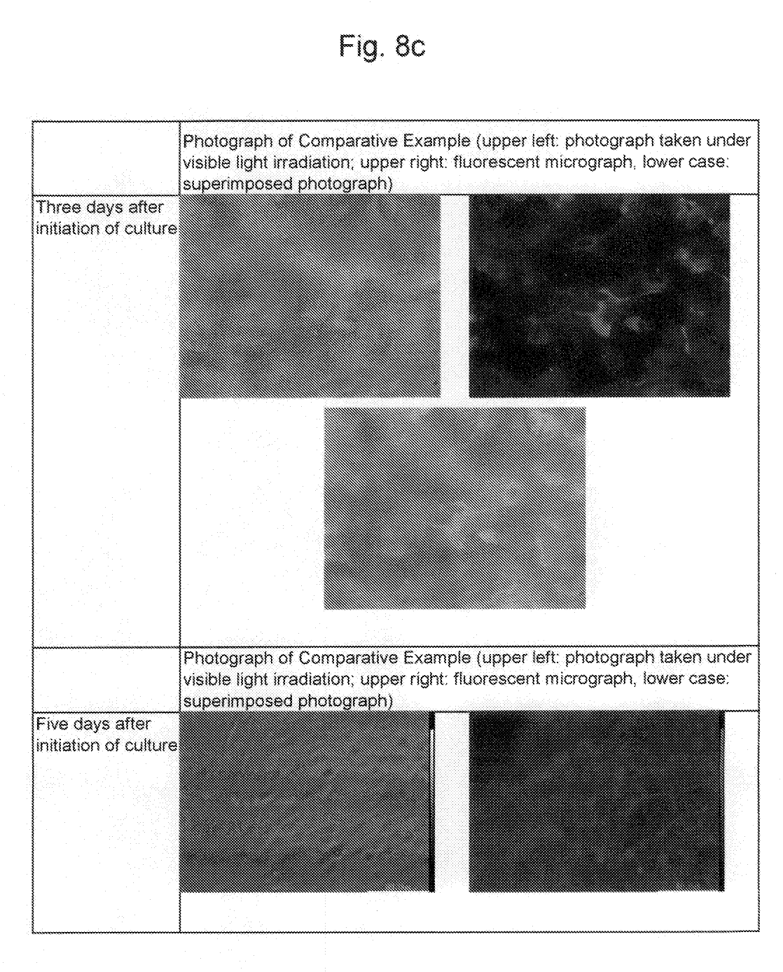

ECM was observed by a fluorescent microscope. The observation results in Examples 1 and 5 and Comparative Example are shown in FIGS. 8a, 8b and 8c, respectively.

In FIG. 8a, the photograph on the upper left side of “3 days after initiation of culture” is that of the cell sheet (adhering onto a substrate) taken from the top under visible light irradiation. The grid, which was slightly seen in the photograph is the outline of a grid-form groove (depressions). The photograph on the upper right side of “3 days after initiation of culture” is a fluorescent micrograph of the same cell sheet taken from the same direction (from the top). The photograph at the lower center of “3 days after initiation of culture” is a superimposed photograph obtained by stacking the photograph taken under visible light irradiation (the upper left side) and the fluorescent micrograph (the upper right side).

In FIG. 8a, the photograph on the left side of “5 days after initiation of culture” is that of the cell sheet (adhering onto a substrate) taken from the top under visible light irradiation. The photograph on the right side of “5 days after initiation of culture” is a fluorescent micrograph of the same cell sheet taken from the same direction (from the top). In the fluorescent micrograph of the cell sheet obtained 5 days after culture, the site present on the right side below the line, which crosses the center of the fluorescent micrograph diagonally, is a lift-up portion of the cell sheet removed from the substrate and comes up in the front of the photograph (that is, in the direction perpendicular to the surface of the photograph). On the other hand, whereas the cell sheet present on the left side above the line (line looks bright), which crosses the center of the fluorescent micrograph diagonally, is a portion attached on the substrate. As is apparent from the photographs of “3 days after initiation of culture”, ECM is formed around a cell, and that ECM is formed even above depressions in the same manner as on the top face. As is apparent from the photographs of “5 days after initiation of culture”, ECM is formed over the entire adhesion surface of the cell sheet removed from the substrate and facing the substrate, and that the cell sheet is removed integrally with ECM.

Individual photographs of FIG. 8b are those of cultured cells using the substrate of Example 5 taken in the same conditions as in FIG. 8a. In the micrograph of the cultured cells of “5 days after initiation of culture”, the site present on the bright portion on the upper right side of the fluorescent micrograph is a lift-up portion of the cell sheet removed from the substrate and comes up in the front of the photograph (that is, in the direction perpendicular to the surface of the photograph). As is apparent from the photographs of “3 days after initiation of culture”, ECM is formed around a cell 3 days after initiation of culture also in Example 5, and that ECM is formed even above depressions in the same manner as on the top face. As is apparent from the photographs of “5 days after initiation of culture”, ECM is formed over the entire adhesion surface of the cell sheet removed from the substrate and facing the substrate, and that the cell sheet is removed integrally with ECM also in Example 5.

Individual photographs of FIG. 8c are those of cultured cells using the substrate of Comparative Example taken in the same conditions as in FIG. 8a. In the micrograph of the cultured cells of “5 days after initiation of culture” of FIG. 8c, the site present on the upper left side is a lift-up portion of the cell sheet removed from the substrate and comes up in the front of the photograph. As is apparent from the figures, the formation state of ECM in Comparative Example does not virtually differ from those of Examples 1 and 5.

In short, it has been proved that ECM is formed around a cell similarly not only on the top face of the projections but also above a groove (depressions) of a substrate according to the present invention, and that when a cell sheet is removed, ECM formed on the top face and above the groove (depressions) is transferred integrally with the cell sheet and does not remain on the substrate.

### COMPARATIVE EXAMPLE

Water permeability to a microporous film coated with a temperature responsive polymer In this Comparative Example, a microporous film (described in O. H. Kwon, J. Biomed. Mater. Res., (2000) Apr.; 50(1):82-9) having a temperature responsive polymer (polyisopropylacrylamide) which is graft-bonded thereon was checked for permeability by a culture solution.

A microporous film 1 shown in Table 3 was charged with oxygen under a reduced vacuum of about 60 to 150 mm Torr, treated with a plasma discharge at 400 W for 3 minutes, coated with a coating composition, which was prepared by dissolving a prepolymer (polyisopropylacrylamide (Aldrich Article No. 535311) having a molecular weight of 20,000 to 25,000 and commercially available from Aldrich) in a content of 1 wt % and an isopropyl acrylamide monomer in a content of 40wt % in isopropyl alcohol, once irradiated with electron beams at a dose of 15 Mrad, washed and dried. In this manner, the microporous film 1 coated with a temperature responsive polymer was obtained. For comparison, a microporous film 1 (base film) not coated with the temperature responsive polymer was prepared. These films were set at a suction holder made of glass and having a filter of 47 mm in diameter. The suction holder made of glass was placed in 1 L of a suction bottle connected to an aspirator. Filtration was performed by alternately supplying distilled water of 20° C. and 40° C. As a result, as shown in Table 4, both warm water and cold water passed though the base film not coated with a temperature responsive polymer, whereas only warm water passed through the microporous film 1 whose surface was coated with a temperature responsible polymer but cold water did not virtually pass through the microporous film.

Microporous films 2 to 6 shown in Table 3 were coated with a temperature responsive polymer and checked for water-permeability, in the same manner as above. As a result, as is the same as in the results of the microporous film 1 shown in Table 4, it was demonstrated that the water permeability of these films decreases in low temperature conditions at which a cell sheet is removed.

TABLE 3

| | Name of film | Manufacturer | Article No. |
|---|---|---|---|
| Microporous film 1 | Microporous polyethylene | Tokuyama Corporation | Porum PH500 |
| Microporous film 2 | Microporous polypropylene | Tokuyama Corporation | NF Sheet NN100 |
| Microporous film 3 | Microporous polyethylene | Asahi Kasei Chemicals | Highpore NB 630 |
| Microporous film 4 | Microporous polyethylene | Asahi Kasei Chemicals | Highpore H6022 |
| Microporous film 5 | Microporous polycarbonate | Whatman | Cyclopore 7060-4704 |
| Microporous film 6 | Microporous polycarbonate | Whatman | Cyclopore 7060-4713 |

TABLE 4

| | Permeation | |
|---|---|---|
| | amount of | Permeation |
| | cold water | amount of warm |
| | in 2 minutes | water in 2 minutes |
| Base film | 4.9 mL | 5.0 mL |
| Microporous film coated with | 0.3 mL | 5.1 mL |
| temperature responsive polymer | | |

The entire disclosure of the publications, patents and patent applications cited in this specification is incorporated herein by reference.

What is claimed is:

1. A cell culture substrate for forming a cell sheet by culturing cells, comprising a plurality of projections each having a top face and depressions formed between the projections, wherein the depressions each have an opening whose dimensions are too small for a cell to be cultured to enter.

2. The cell culture substrate according to claim 1, wherein the top face is coated with at least one type of polymer selected from the group consisting of a temperature responsive polymer, pH responsive polymer and ion responsive polymer.

3. The cell culture substrate according to claim 1, wherein the dimensions of the opening of each of the depressions are smaller than outer dimensions of a cell to be cultured.

4. The cell culture substrate according to claim 1, wherein a total area of the openings of the depressions is 10 to 60% of the sum of a total area of the top faces and a total area of the openings of the depressions.

5. The cell culture substrate according to claim 1, wherein the opening of each of the depressions has a width of 0.1 to 10 μm.

6. The cell culture substrate according to claim 1, wherein the opening of each of the depressions has a depth of not less than 0.01 μm.

7. The cell culture substrate according to claim 1, wherein the dimensions of the top face are equal to or larger than outer dimensions of a cell to be cultured.

8. The cell culture substrate according to claim 1, wherein the width of the top face is 1 to 20 μm.

9. The cell culture substrate according to claim 1, wherein the depressions form a grid-form groove.

10. The cell culture substrate according to claim 1, wherein the depressions form a plurality of linear grooves.

11. A method of forming a cell sheet comprising by culturing cells by use of the cell culture substrate according to claim 1.

12. A cell sheet prepared by the method of claim 11.
